(19) United States
(12) Patent Application Publication
(10) Pub. No.: US 2018/0032696 A1
(43) Pub. Date: Feb. 1, 2018

(54) WHOLECARE

(71) Applicant: Nancy ROME, Baltimore, MD (US)

(72) Inventor: Nancy ROME, Baltimore, MD (US)

(21) Appl. No.: 15/661,997

(22) Filed: Jul. 27, 2017

Related U.S. Application Data
(60) Provisional application No. 62/367,987, filed on Jul. 28, 2016.

(51) Int. Cl. G06F 19/00; A61B 5/00; G06Q 10/10

(52) U.S. Cl. CPC G06F 19/3418; G06Q 10/1093; A61B 5/6802

(57) ABSTRACT

WholeCare is a holistic communications hub for all those involved in looking after one person or patient. The system streamlines and facilitates communication among the family, doctors and caregivers, so stress is lessened and time saved for all parties, and quality of life is improved for the person who needs care. WholeCare's platform and approach addresses widespread inconsistencies in communication. As healthcare needs grow in complexity and cost, WholeCare will meet the needs of the elderly living at home by engaging their families and caregivers in a unique, efficient, user-friendly and comprehensive manner.

### CROSS-REFERENCE TO RELATED APPLICATIONS

This application claims benefit of Provisional Application No. 62/367,987 filed Jul. 28, 2016, incorporated herein by reference.

### STATEMENT REGARDING FEDERALLY SPONSORED RESEARCH OR DEVELOPMENT

None.

### FIELD

This technology relates to computer systems and methods for managing information and tasks, and more particularly to computer-assisted systems and methods for managing and coordinating personalized care of an individual. Still more particularly, example non-limiting technology relates to holistic communications hubs and associated supporting infrastructure for all those involved in looking after one person or patient.

### BACKGROUND AND SUMMARY

The U.S. population of adults age 65 and older was approximately 44 million in 2010, but will be over 88 million in 2050. Given a choice most elderly in the U.S. prefer to live at home instead of in a nursing home or other institutional settings. There are clear health benefits to staying at home and connecting with their communities. Civic engagement and volunteering can reduce mortality; increase physical function, muscular strength, and levels of self-rated health; reduce symptoms of depression and pain; and increase life expectancy.

However, caring for the elderly at home increasingly falls to family members providing “free” care, which can have enormous hidden costs. In 2013, AARP estimated the aggregate economic value of families providing free home care to elderly relatives was $470 billion annually. There are hundreds of articles about our sandwich generation facing raising children and caring for our parents. A recent blog post from the NYTimes discusses how “aging in place” is the new model for looking after our aging population. Unfortunately, much of our existing health care system is still geared toward institutionalization and professional services. The caregiver of an aging individual whose goal is to care for the person in the home often feels like a trailblazer. Lots of resources are available, but finding and combining them into a safe, effective and cost-effective solution can be challenging.

Many caregivers' apps are on the market already, but sharing of information among the various parties is silo'ed usually—or the apps provide for only a small part of what's needed, but are not deep enough or extensive enough. Often, the caregiver apps are merely a way to find caregivers—or to keep track of paying them. They do not help the caregiver themselves render care and coordinate with health care professional, family and others.

There are also apps available for keeping the caregiver's lives less stressful—but again, this is only part of what's so badly needed.

There is a need to streamline and facilitate communication among the family, doctors and caregivers, so stress is lessened and time saved for all parties, and quality of life is improved for the person who needs care.

### BRIEF DESCRIPTION OF THE DRAWINGS

The detailed description of exemplary non-limiting illustrative embodiments is to be read with the drawings, of which:

FIG. 1 shows a home page;

FIG. 1A shows a noon page—calendar/schedules;

FIG. 2 shows a 1 o'clock page—messaging;

FIG. 3 shows a 3 o'clock page—vitals;

FIG. 4 shows a 5 o'clock page—nutritional and menus;

FIG. 5 shows a 6 o'clock page—medication and supplements;

FIG. 6 shows a 7 o'clock page—important numbers and team contacts;

FIG. 7 shows a 9 o'clock page—activities, entertainment, exercise, therapy, visitors, appointment, likes, dislikes, special instructions;

FIG. 8 shows an 11 o'clock page—house, apartment, quirks, how things work, household schedule-linens, etc.; and

FIG. 9 shows an example non-limiting system block diagram.

### DETAILED DESCRIPTION OF EXAMPLE NON-LIMITING IMPLEMENTATIONS

Many apps currently available identify themselves as “caregiver apps.” However, most are geared toward billing, scheduling and administrative tasks. WholeCare's platform and approach is completely different. We are addressing widespread inconsistencies in communication. As healthcare needs grow in complexity and cost, WholeCare will meet the needs of the elderly living at home by engaging their families and caregivers in a unique, efficient, user-friendly and comprehensive manner.

WholeCare is a holistic communications hub for all those involved in looking after one person or patient.

WholeCare provides access for the patient's family, doctors (HIP AA compliant, with various differentiated levels of security), nursing agencies, therapists and caregivers—anyone involved (and invited) in looking after one person.

There are several versions—one for nursing homes or hospice and one for home care.

WholeCare also makes health care insurance more efficient—especially long-term health care insurance.

One can build and change for those looking after babies, children, those who need therapy or personal training, or even such diverse applications as pets, farming and gardening where the object of the care is not a person.

Nursing homes and Hospice—could use a version of it to log into—but should be portable. The system can be altered & tailored specifically for nursing homes, etc., to tie into their systems, but the first primary focus for some non-limiting implementations is caring for someone at home/privately or in a modified form for those living independently. But will be easily expandable and invaluable for any age.

The app can (upon client's approval) export information/gathered data to healthcare insurers or long-term health care insurers/certifying agencies, etc.—so to avoid the embarrassing and humiliating questions/interviews which need to take place every six months, sometimes to keep long term care coverage. This will also a money-saver for agencies, possibly saving them manpower and home visits.

A new kind of extensive chart is provided—one not however restricted to the conventional contents of medical charts, nor should it need to include EMR-only when required by physicians.

Rather, it's a holistic approach to keep track of the “whole” person in question, which can be logged into or otherwise accessed by all concerned parties.

There are many possible applications. The system makes it easy for everyone looking after one person to stay in touch with each other and on top of the case. It can also be used for a farm or second home to monitor on-going caretaking.

Another feature may need to be a log-in (time card) for caregivers—so they are prompted to fill in food, drink, activity, meds, etc.

Easy to use, visually arresting but VERY simple, almost game-like for users, despite the multiple functions. No one need toil away endlessly entering information, but instead can use touch and taps and screen swipes to keep track of what happens in the person's life each day.

Apart from a mobile app, there are two desktop components and associated interfaces—one for the primary person in charge of running the healthcare team (probably a family member), and one for the doctors and other professionals involved. Given online onerous tasks doctors must manage, it's possible they would prefer a simpler mobile app, where they can check in on many patients.

One aspect of the technology herein is to build an integrated software platform and mobile communications hub making living at home a simple, worry-free, and cost-effective option for the elderly, their families, and caregivers.

Another aspect is to provide a vital resource and help for those who never expected to be a caregiver.

A further aspect is seamlessly engage patients, family members, caregivers, health care providers and all others involved in home care, so everyone is on the same page at all times, and in real time by organizing and simplifying schedules and information; facilitating trusted communication among authorized users; and, most importantly, individualizing the care experience.

Other non-limiting features and advantages include:
Individualized care by ensuring that the person being cared for is appreciated and understood and that their story is available.
Tell the story of the person being cared for using modern technology.
Employ an intuitive user interface for caregivers to track activities, medication, vitals, nutrition, and other data using taps and swipes, with minimal input.
Provide an integrated scheduling and calendar tool with notes and alerts for important tasks and recurring events to connect caregivers, family and providers.
Limit caregiver error; improve efficiency; reduce hospital visits, admissions and re-admissions; and improve clinical outcomes.
Enable retrieval of health data by doctors and insurance providers—as needed.
Enhance the wellbeing of caregivers through self-assessment, self-care and community supports.
Provide HIPAA-compliant, blockchain protected security for records and trusted communication among authorized users.
Simplify home care and substantially reduce the on-going financial and emotional costs and stress of health care for patients and families.
Create a healthier environment for the elderly who choose to live at home.

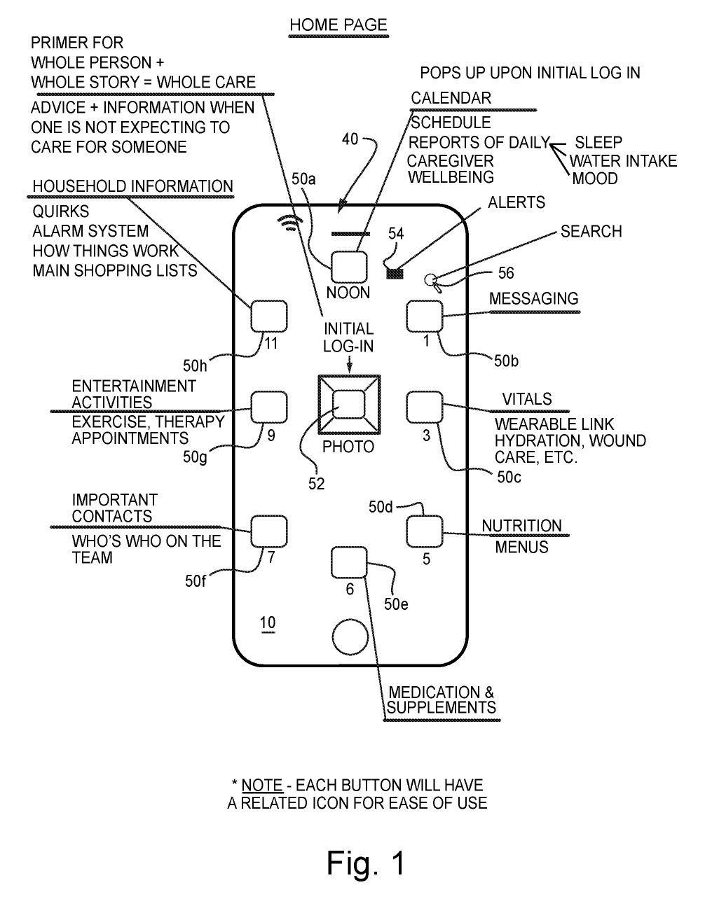

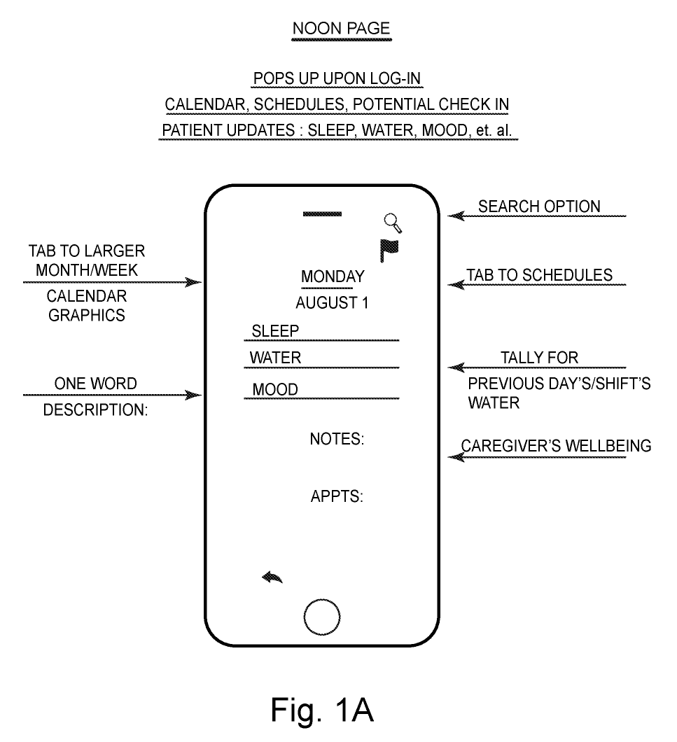

In more detail, FIG. 1 shows an example non-limiting home page user interface 40 displayed on the touch screen 10 of a smart phone or other suitable appliance 202. The home page user interface 40 includes several user-actuatable icons or other buttons that, when depressed or otherwise selected by a user, initiate corresponding functions. In this example, buttons 50a-50h are arranged around the periphery of the user interface, but other examples could arrange them differently such as in columns or any other suitable arrangement. Each of buttons 50a-50h may be displayed using the corresponding graphically-distinctive icon for ease of recognition and access.

In the center of the home page user interface 40, a photo 52 of the patient being cared for may be displayed. Depressing the photo to select it may initiate an initial log-in that challenges the user to respond with security information to protect confidentiality. An alert icon 54 may change state when alerts are present. A search icon 56 may be depressed to provide a text or voice actuated search function for particular user-specified information.

In the particular example shown, buttons or icons 50a-50h correspond to these function initiations described below:

A button 50a at the “noon” or top center position of the user interface may pop up upon initial log-in and provide access to a calendar and scheduling function including imports of daily sleep, water intake, mood, etc. (see FIG. 1A). It also invokes a “caregiver wellbeing” function.

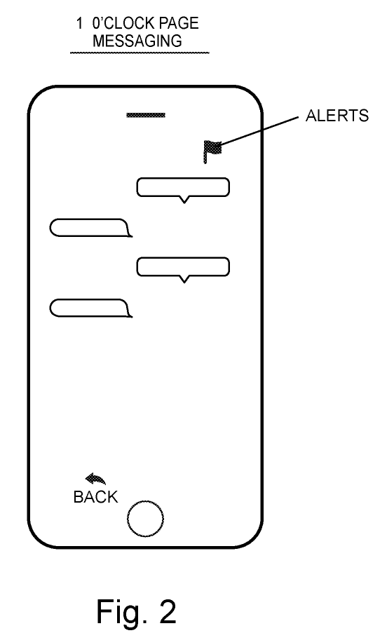

A button 50b at the “1 o'clock” position may initiate a function to provide messaging (see FIG. 2).

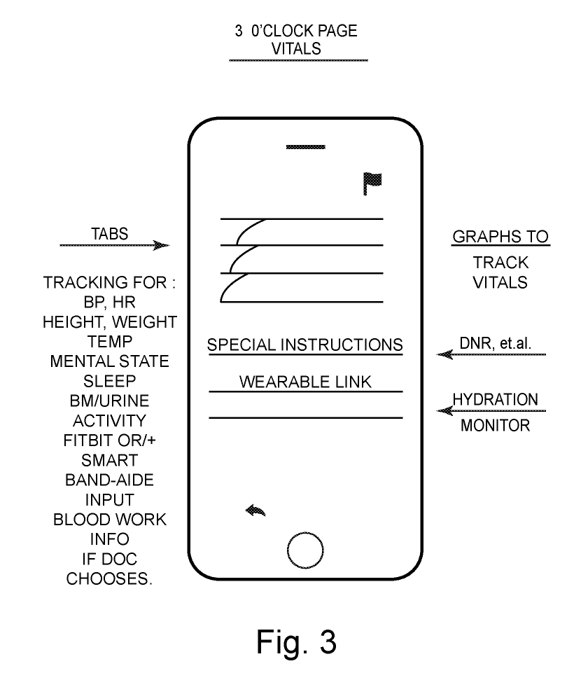

A button 50c at the “3 o'clock” position may initiate a function to record, track and report vital signs such as heart rate, blood pressure and blood work (see FIG. 3). This may include a wearable link to measure hydration, wound care, etc.

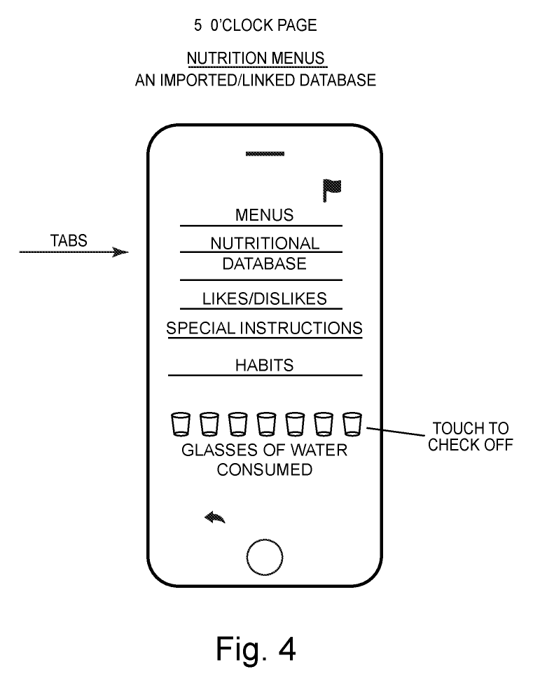

A button 50d at the “5 o'clock” position may report on nutrition, menus (see FIG. 4).

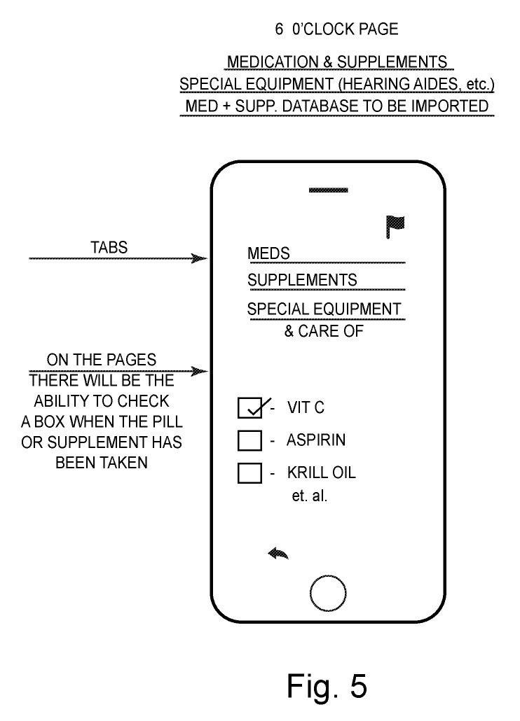

A button 50e at the “6 o'clock” position may report on medication and supplements (see FIG. 5).

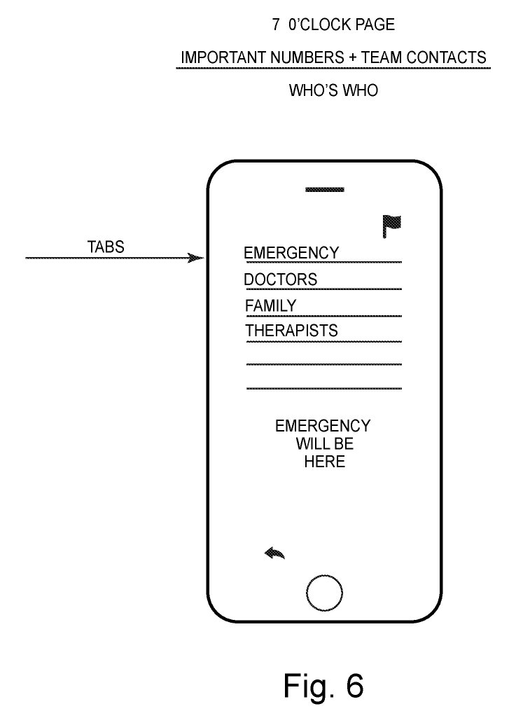

A button 50f at the “7 o'clock” position may report important contacts such as providing a who's who on the care team (see FIG. 6).

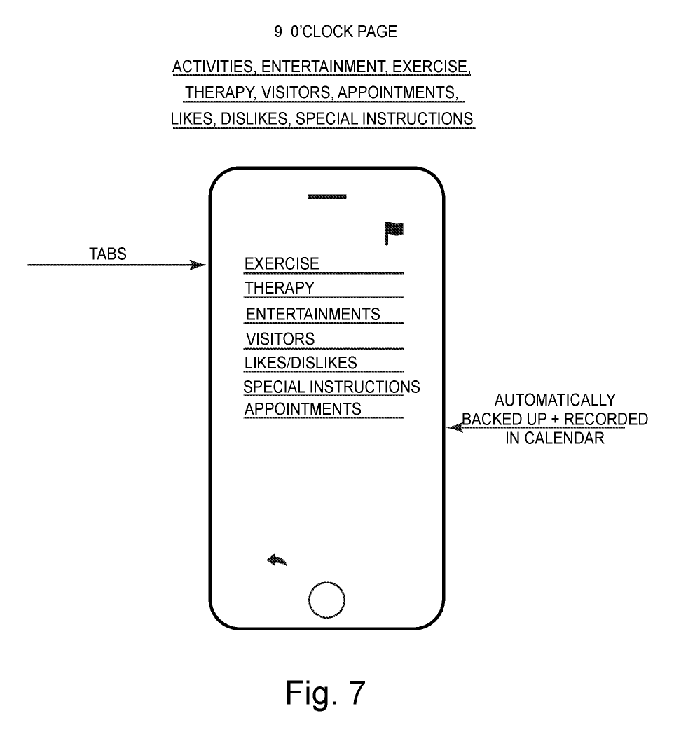

A button 50g at the “9 o'clock” position may report and track entertainment activities such as exercise, therapy appointments, etc. (see FIG. 7).

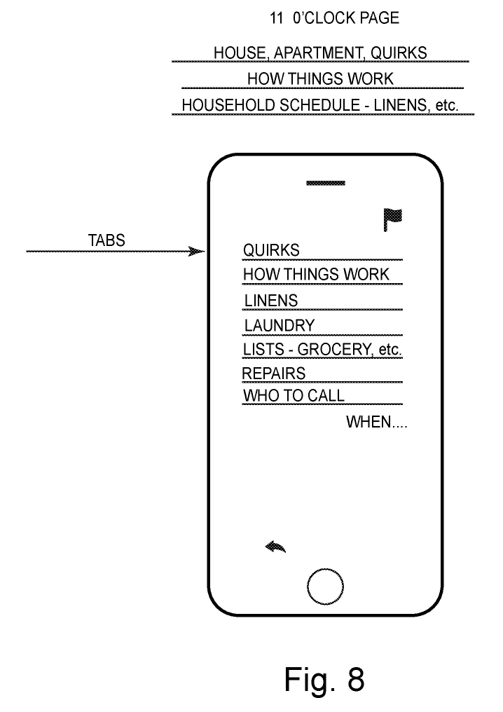

A button 50h at the “11 o'clock” position may report household information such as quirks, how to operate the home alarm system, how the dishwasher or microwave work, tracking a main shopping list, etc. (see FIG. 8).

Upon depressing each of the buttons 50a-50h, a corresponding one or more additional screens may be displayed to replace the home page user interface 40 in FIG. 1. Upon completing the operations of the more detailed invoked function, the user interface 40 may return to the home page to enable the user to access a different function.

To begin with, think of the home page user interface 40 of FIG. 1 as the face of a clock, with the patient at the center. At the center “button” 52 will be link to a description of the patient and all vitals, important history, etc., with a summary of likes, dislikes, habits, etc.—also repeated under some of the other pages.

The hours of the clock face are links for all the parties to use and for information pertaining to each element involved in caregiving—each with its own main page with tabs for subsequent pages.

An example user interface may contain:

Access to a primer (through the main button 52 about the person)—about the caregiving task at hand—What to Expect When You're NOT Expecting [to do this], i.e., a summary of the patient's medical background including e.g., blood work (NOT including electronic medical records, unless deemed critical by the patient's doctors), and WHO that person at the center of all this IS—were they the US Senator from Maryland between 1972 and 1978 . . . where they're from, family history—so the caregiver can converse in an intelligent way and not operate in a vacuum, not knowing the person.

As part of this primer, there can be some accumulated information and advice on how better to care for someone in many situations—a “Martha Stewart”™ or concierge f caregiving.

There is no need for an actual clock face—this is merely a way to envision the links on the home page and how they might appear graphically:

NOON: (FIG. 1A)

Log-In—Links/passwords for the household/Wi-fi, etc., will be set up for family members, doctors who need access.

As indicated, several levels of security will need to be built in.

Calendar/daily chart/diary 50a:

The caregiver should see on the calendar which would open automatically—hours of sleep, water consumed and state-of-mind—and any other alerts would also show up, gathered from the other “pages” of the app. Some of this information (water, sleep, state-of mind will also be repeated elsewhere—but linked to the calendar for ease of use).

NOTE: There is also data and tracking of vitals, water intake, meal consumption, hours of sleep, state of mind, et. al.—so patterns can be understood and any alterations in those patterns noticed and addressed—via simple but accumulating graphs.

Alerts 54 are also built into each page where appropriate.

There is also a monitoring function for caregiver wellbeing.

1 o'clock Messaging button 50b (FIG. 2):

Messaging—able to link to nursing agencies and to patient's doctor if need be. This can be shared among caregivers—i.e., different nurses at an agency can log in and always be on the same page, which should result in fewer errors and less confusion—a more cohesive way to look after someone. This can be similar to a Caregiver's WhatsApp.

3 o'clock (FIG. 3):

VITALS: State of mind/well-being, degree of dementia, etc. Or simple mood: cheerful, depressed, bored, lonely, weepy, sentimental, confused, etc. episodes (TIAs etc.)

Weight, Height, etc.—Temperature, BP (AU Vitals)—again, a baseline should be established and there will be a place for daily entries in the event of for spikes/anxiety/TIA, etc.

Hours of sleep and naps, BM/urine activity—where appropriate

Updates of vitals can also be tracked via FitBit or automatic monitoring sensing 204. There is the possibility of linking a “smart band-aid” being tested in hospitals, a hydration monitor, or other wearable link. A smart phone based CT scanner to monitor bed sores, surgery sites, wounds, etc., can also be incorporated.

Recent bloodwork is also recorded and displayed here if the health care provider chooses.

Certified DNR instructions, if they apply—also should be repeated under Contact List page so that it's clear who the Healthcare Proxy is.

5 o'clock Nutrition/Menus button 50d (FIG. 4):

Menus and Calories [A nutritional database is imported in one non-limiting embodiment.] (% of meals consumed)—should be able to make up for missing vitamins/minerals . . . target calories can be entered with required foodstuffs. If not say enough Vit C has been consumed—there's a little alert 54 for the caregiver . . . in this section can be favorite foods, etc. Likes—foods, drinks, etc.—but these will also be noted in the nutrition section. Dislikes—ditto Habits (bad and good) Water intake—by glass, bottles, etc., again, minimum should be tracked—using simple and easy-to-use graphics of little images of glasses that can be tapped to record intake. This will be linked to register in the Calendar.

6 o'clock Medication and Supplements button 50e (FIG. 5):

Pills/meds/vitamins/minerals/oils—including import of a database for this and a way of checking off what's been consumed.

Alerts 54 for missed meds, etc., is also part of this.

Special needs: Hearing aids, dental bridge, etc. and care of such items.

7 o'clock Important Contacts button 50f (FIG. 6): Important Numbers:

Emergency numbers and Contact List: family, friends, doctors etc.—contact base upload, etc., and a photo link for all smart phones (considering possible video link as well). A Who's Who for all involved.

9 o'clock Entertainment Activities button 50g (FIG. 7): Activities:

Exercise—length of time, work out, weights, walking, stairs

Visitors—this might go with separate notes at the end of each day

Massages—a crucial part to keep patient healthy—if there are sufficient funds

Shampoos, manicures, podiatrist etc.—how often, where, etc.

Doctor's appointments and listing of doctors/specialists and emergency numbers (legal counsel, etc.), ways to get hold of family members, POAs

Likes—foods, drinks, etc.—but these will also be noted in the nutrition section. Dislikes—ditto Habits (bad and good) Entertainment—does the patient respond to listening to the news or prefers Mozart to the golf or tennis channel—Radio or TV, etc.

11 o'clock Household Information button 50h (FIG. 8):

House (where applicable) Quirks, Information, etc.: A whole section can be added to keep everyone abreast of specifics about location, care of house, etc. Repairs needed, leaks, house quirks, etc., workmen, etc.

Simple Information for Linens:

Sheets and Towels—when they get changed—with note/box to tick when changed. This should be linked to the calendar.

How things work in the house, sticky doors, tricky locks and windows, alarm system, housekeeper? Property care and maintenance, etc. Links to the contact list for repairmen, etc.

Grocery/Household Main List: Kept so that shopping and stocking up is simplified and centralized, so that when things run out—no one is running circles at the last minute trying to shop.

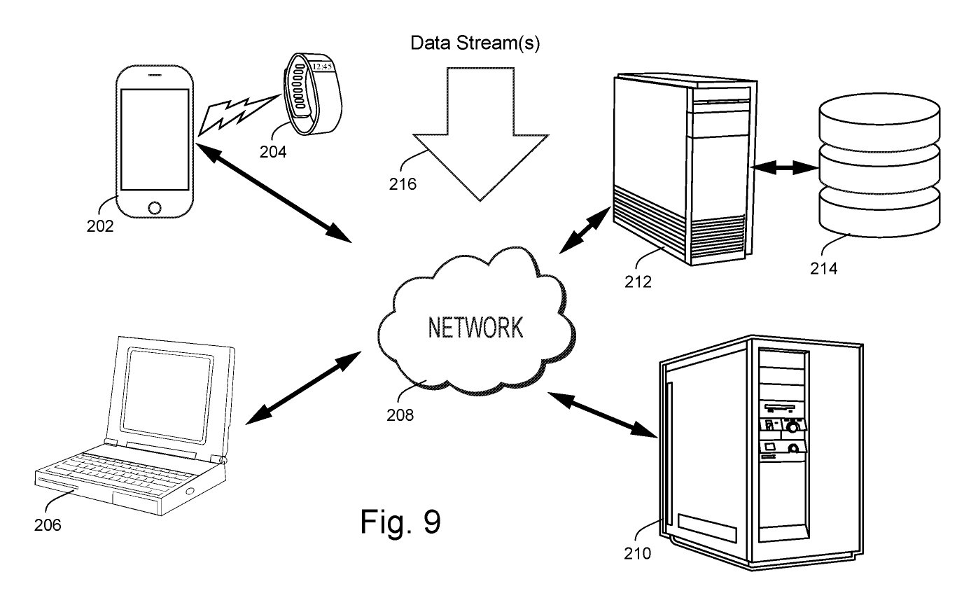

FIG. 9 shows an example block diagram of a non-limiting implementation of a system for implementing WholeCare as described above. In this example, the user appliance 202 such as a smart phone or any other processor-based computing or terminal interface device may communicate with various sensors and other inputs including for example a wearable or other vital gathering device 204 (e.g., watch, patch, etc.) that monitors the patient's heart rate, hydration level, skin temperature, sleep patterns, etc. Additional inputs may be provided from home security systems, cameras, temperature sensors and other Internet of things devices or enhanced patient monitoring

The user appliance 202 may communicate via a network 208 with a server 212 that can access one or more databases 214. Some of the functionality described above may be implemented as computer code stored in non-transitory memory on the user appliance 202, whereas other functions may be implemented as computer code executing on non-transitory memory disposed in the “cloud” on a server 212, as understood by those of ordinary skill in the art.

Other user appliances such as a laptop computer 206, a conventional interactive voice response system, or any other user interface may also access server 212 via network 208. One or more additional servers 210 such as those operated by hospitals, health care providers, or the like may interconnect with server 212 to provide by directional information exchange. Data streams 216 may, via network 208, provide additional inputs such as videos, documents, images or other updates to server 212 for storage into databases 214 and selective providing to user appliance 202.

The system in FIG. 9 is preferably designed to be highly scalable to support many user appliances 202 and associated patients. Well known security measures implemented on server 212 provide highly secure compartmentalization of patient information while sharing general resources such as health care provider directories, home repair directories, etc. Conventional communications infrastructure such as Internet, cellular data communications and other connectivity may provide information exchange between the FIG. 9 components.

While the invention has been described with what is presently considered the most practical and preferred embodiments, it is to be understood that the invention is not to be limited to the disclosed embodiments, but on the contrary, is intended to cover various modifications and equivalent arrangements within the spirit and scope of the appended claims.

1. A system for monitoring at least one patient comprising:
a portable device having a processor, a memory and a communications circuit; and
a server in communication with the portable device, the server including a processor, a memory and a communications circuit,
wherein the server receives information relating to the patient's health and/or care from the portable device and provides information relating to the health status of the patient to the portable device;
the portable device providing a user interface offering at least four of the following functions:
(i) access to a calendar and scheduling function including imports of daily sleep, water intake, mood;
(ii) a caregiver wellbeing function/tracker.
(iii) messaging,
(iv) record, track and report vital signs such as heart rate, blood pressure and blood work,
(v) a wearable link to measure hydration and/or wound care, and vitals.
(vi) report on nutrition and menus,
(vii) medication and supplements,
(viii) report important contacts such as providing a who's who on the care team,
(ix) report and track entertainment activities such as exercise and therapy appointments, and
(x) report household information.

2. The system of claim 1 wherein the portable device comprising a smart phone.

3. The system of claim 1 wherein the user interface comprises a web interface.
